(19) United States
(12) Patent Application Publication
(10) Pub. No.: US 2026/0110683 A1
(43) Pub. Date: Apr. 23, 2026

(54) HISTOTRIPSY SYSTEMS AND METHODS FOR REJECTED ORGAN RECOVERY

(71) Applicant: HistoSonics, Inc., Plymouth, MN (US)

(72) Inventor: Joshua STOPEK, Minneapolis, MN (US)

(21) Appl. No.: 19/361,539

(22) Filed: Oct. 17, 2025

Related U.S. Application Data
(60) Provisional application No. 63/708,652, filed on Oct. 17, 2024.

(51) Int. Cl. G01N 33/50

(52) U.S. Cl. CPC G01N 33/5091

(57) ABSTRACT

A system and method for rendering a diseased organ implant eligible, the method including acquiring an image data set of a potential donor organ, identifying one or more disease states in the potential donor organ, wherein the one or more disease states render the organ ineligible for transplant, harvesting the potential donor organ, applying histotripsy therapy, and confirming elimination/reduction of the disease state in the potential donor organ.

### PRIORITY CLAIM

This patent application claims priority to U.S. Provisional Application No. 63/708,652, titled “REJECTED ORGAN RECOVERY” and filed on Oct. 17, 2024, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure is directed to systems and methods of assessing tissues and organs having one or more features that would otherwise disqualify them from being employed in an organ or tissue replacement surgery, treating the organs or tissues with high intensity therapeutic ultrasound (HITU), assessing the efficacy of the treatments in eliminating the disqualifying feature, and when determined adequately treated making the organs or tissues available for implantation in needy patients.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, histotripsy realm; it is necessary to deliver acoustic energy in the form of high amplitude acoustic pulses with low duty cycle.

Compared with conventional focused ultrasound technologies, histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) histotripsy produces lesions in a controlled and precise manner. It is important to emphasize that unlike thermal ablative technologies such as microwave, radiofrequency, high-intensity focused ultrasound (HIFU), cryogenic, or radiation, histotripsy relies on the mechanical action of cavitation for tissue destruction and not on heat, cold or ionizing energy.

### SUMMARY

One general aspect of the disclosure includes a method of rendering a diseased organ implant eligible. The method also includes acquiring an image data set of a potential donor organ; identifying one or more disease states in the potential donor organ, where the one or more disease states render the organ ineligible for transplant; harvesting the potential donor organ; applying histotripsy therapy; and confirming at least one of elimination or/reduction of the disease state in the potential donor organ. Other embodiments of this aspect include corresponding computer systems, apparatus, and computer programs recorded on one or more computer storage devices, each configured to perform the actions of the methods.

Implementations may include one or more of the following features. The method where the histotripsy therapy is applied prior to harvesting of the potential donor organ. Histotripsy therapy is applied after harvesting of the potential donor organ. The method may include performing perfusion on the potential donor organ prior to or during the application of the histotripsy therapy. The metho may include assessing one or more blood biomarkers to assess the efficacy of the histotripsy therapy to confirm the at least one of elimination or reduction of the disease state. Applying histotripsy therapy skeletonizes a portion of the potential donor organ. The skeletonization reveals one or more blood vessels or ducts within the potential organ. The method may include ligating the one or more blood vessels or ducts within the potential organ. The method may include determining that a sub-portion of the potential donor organ, skeletonized, and ligated from the potential donor organ is suitable for transplant. The histotripsy therapy is applied to a tumor or cyst. At least one of the mechanical properties or/integrity of the tumor or cyst are reduced following application of histotripsy therapy. Application of histotripsy therapy triggers an immuno-response. The method may include monitoring blood biomarkers to assess a magnitude of the immuno-response. Confirming the at least one of elimination or/reduction of the disease state in the potential donor organ is based on a change in the blood biomarkers. The method may include acquiring the blood biomarkers before application of histotripsy therapy and after application of at least one histotripsy therapy. Confirming at least one of elimination or reduction of the disease state in the potential donor organ is based on a comparison of the image data set and a second image data set. An artificial intelligence or a trained neural network performs the comparison of the image data set and the second image data set. The potential donor organ is selected from the group may consisting of a liver, a kidney, bone marrow, a lung, a pancreas, an esophagus, an eye, a pituitary gland, a trachea, skin, and blood. The disease state includes one or more of cancerous tumors, cirrhosis, cysts, fatty deposits, or blood cancers. The histotripsy therapy is applied to a diseased portion of the potential donor organ. The histotripsy therapy is applied to a non-diseased portion of the potential donor organ. The histotripsy therapy is applied to a diseased portion of an organ other than the potential donor organ. The method may include implanting the potential donor organ in a patient in need thereof. Implementations of the described techniques may include hardware, a method or process, or computer software on a computer-accessible medium.

### BRIEF DESCRIPTION OF THE DRAWINGS

The features of the disclosure are set forth with particularity in the claims that follow. A better understanding of the features and advantages of the disclosure will be obtained by reference to the following detailed description that sets forth illustrative embodiments, in which the principles of the disclosure are utilized, and the accompanying drawings of which:

FIG. 1A is a perspective view of a histotripsy system in accordance with the disclosure;

FIG. 1B depicts a therapy transducer and imaging transducer in accordance with the disclosure;

FIG. 2 depicts a histotripsy system deployed to treat a patient in accordance with the disclosure;

FIG. 3 is a plot of a pressure wave during application of a histotripsy ultrasound pulse in accordance with the disclosure;

FIG. 4 is a flow diagram of a method of employing histotripsy to remove diseased tissue from an organ to render an organ eligible for transplantation in accordance with this disclosure; and

FIG. 5 is a flow diagram of another method of employing histotripsy to remove diseased tissue from an organ to render an organ eligible for transplantation in accordance with this disclosure.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing, and ablation. Furthermore, due to tissue selective properties, histotripsy may be used to create a cytoskeleton that allows for subsequent tissue regeneration either de novo or through the application of stem cells and other adjuvants. Histotripsy can also be used to cause the release of delivered agents such as chemotherapy and immunotherapy by locally causing the release of these agents by the application of acoustic energy to the targets. As will be described below, the acoustic cavitation system may include various sub-systems, including a cart, therapy, integrated imaging, robotics, coupling and software subsystems. The acoustic cavitation system also may comprise various other components, ancillaries, and accessories, including but not limited to computers, processors, memory, software, applications, cables, connectors, networking devices, power supplies, displays, drawers/storage, doors, wheels, and various simulation and training tools, etc. All systems, methods and means of creating/controlling/delivering histotripsy are considered to be a part of this disclosure.

### The System

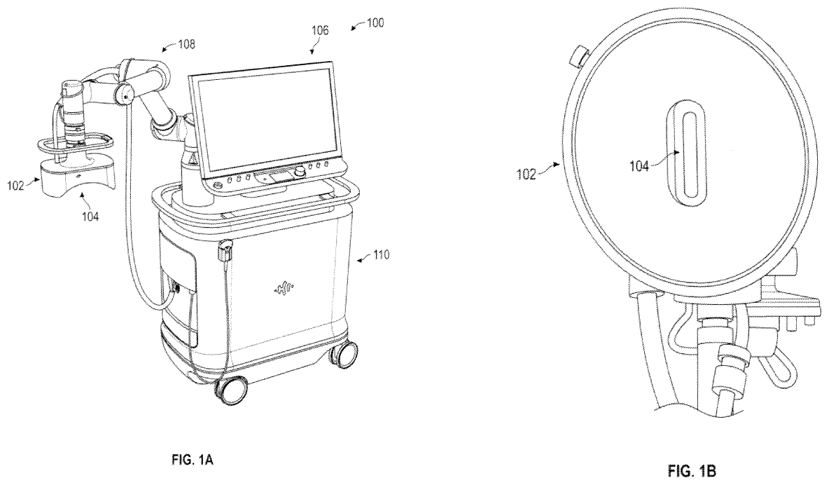

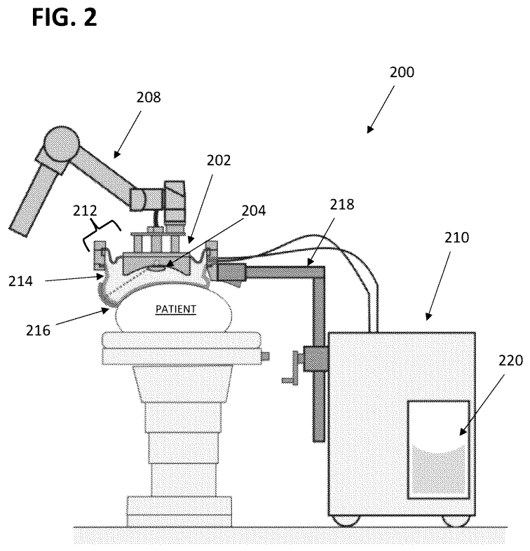

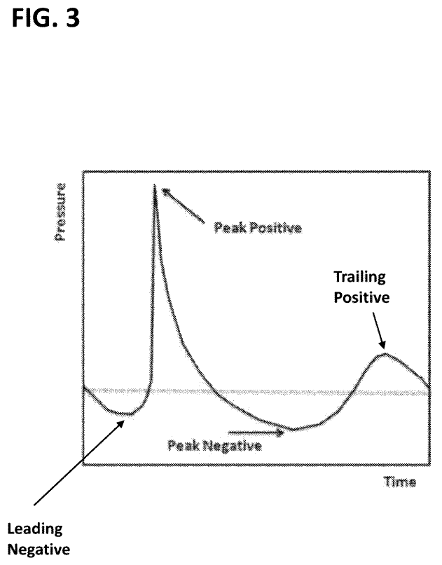

FIG. 1A depicts a histotripsy system 100 in accordance with the disclosure. The histotripsy system 100 includes a therapy transducer 102, an imaging system 104, a display and control panel 106, a robotic positioning arm 108, and a cart 110. The system can further include an ultrasound coupling interface and a source of coupling medium (FIG. 2).

FIG. 1B is a bottom view of the therapy transducer 102 and the imaging system 104. As shown, the imaging system 104 can be positioned in the center of the therapy transducer 102. However, other embodiments can include the imaging system 104 positioned in other locations within the therapy transducer 102, or even directly integrated into the therapy transducer 102. In some embodiments, the imaging system is configured to produce real-time imaging at a focal point of the therapy transducer 102. The system also allows for multiple imaging transducers 104 to be located within the therapy transducer 102 to provide multiple views of the target tissue simultaneously and to integrate these images into a single 3-D image.

The histotripsy system 100 may comprise, and the cart 110 may enclose, one or more of various sub-systems, including a therapy sub-system that can create, apply, focus and deliver acoustic cavitation/histotripsy through one or more therapy transducers 102, an integrated imaging sub-system (or connectivity thereto) allowing real-time visualization and display of the treatment site and histotripsy effect through-out the procedure (e.g., via, a robotics positioning sub-system to mechanically and/or electronically steer the therapy transducer) and further enabled to connect/support or interact with a coupling sub-system to allow acoustic coupling between the therapy transducer 102 and the patient, and software to communicate, control and interface with the system and computer-based control systems (and other external systems) and various other components, ancillaries and accessories, including one or more user interfaces and displays, and related guided work-flows, all working in part or together. The histotripsy system 100 may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described in greater detail below, the cart 110 may be configured and arranged to be used in a radiology environment and in some cases in concert with imaging (e.g., computed tomography (CT), cone beam CT and/or magnetic resonance imaging (MRI) scanning). In other embodiments, the cart 110 may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such, depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The cart 110 may also work with a patient surface (e.g., table or bed) to allow the patient to be presented and repositioned in a variety of positions, angles and orientations, including allowing changes to such to be made pre, peri and post-procedurally. The cart 110, and subsystems thereof, may further comprise the ability to interface and communicate with one or more external imaging or image data management and communication systems, not limited to ultrasound, CT, fluoroscopy, cone beam CT, positron emission tomography (PET), PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri, intra and/or post histotripsy) and to provide access to and display of patient medical data including but not limited to laboratory and historical medical record data.

In some embodiments one or more carts may be configured to work together. As an example, one cart may comprise a bedside mobile cart equipped with one or more robotic arms enabled with a therapy transducer, and therapy generator/amplifier, etc., while a companion cart working in concert and at a distance of the patient may comprise integrated imaging and a console/display for controlling the robotic and therapy facets, analogous to a surgical robot and master/slave configurations.

FIG. 2 illustrates one embodiment of a histotripsy therapy and imaging system 200, including a coupling assembly 212. As described above, a histotripsy therapy and imaging system can include a therapy transducer 202, an imaging system 204, a robotic positioning arm 208, and a cart 210.

The therapy and/or imaging transducers are placed in the coupling assembly 212 which can further include a coupling membrane 214 and a membrane constraint 216 configured to prevent the membrane from expanding too far from the transducer. The coupling membrane 214 is filled with an acoustic coupling medium such as a fluid or a gel. The membrane constraint 216 can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 212 is supported by a mechanical support arm 218 which can be load bearing in the x-y plane but allow for manual or automated z-axis adjustment. The mechanical support arm 218 can be attached to the floor, the patient table, or the cart 210. The coupling assembly 212 is designed and configured to conform and hold the coupling membrane 214 in place against the patient's skin while still allowing movement of the therapy/imaging transducer 202, 204 relative to the patient the coupling membrane 214 with the robotic positioning arm 208.

The system can further include a fluidics system 220 that can include a fluid source, a cooling and degassing system, and a programmable control system. The fluidics system is configured for external loading of the coupling assembly 212 with automated control of fluidic sequences so that the coupling membrane 214 can conform around the patient.

### Histotripsy

Histotripsy is achieved by generating short, high amplitude, focused ultrasound pulses to generate a dense, energetic, “bubble cloud,” capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically affect tissue structure, and in some cases liquefy, suspend, solubilize and/or destruct tissue into sub-cellular components.

Histotripsy can be applied in various forms, including: 1) intrinsic-threshold histotripsy which delivers pulses typically with a 1-2 cycles of high amplitude negative/tensile phase pressure exceeding the intrinsic threshold to generate cavitation in the medium (e.g., ˜24-28 MPa for water-based soft tissue), 2) shock-scattering histotripsy which delivers typically pulses 1-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) boiling histotripsy which employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous, and robotically mediated surgical procedures. It may be further targeted, planned, directed, and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

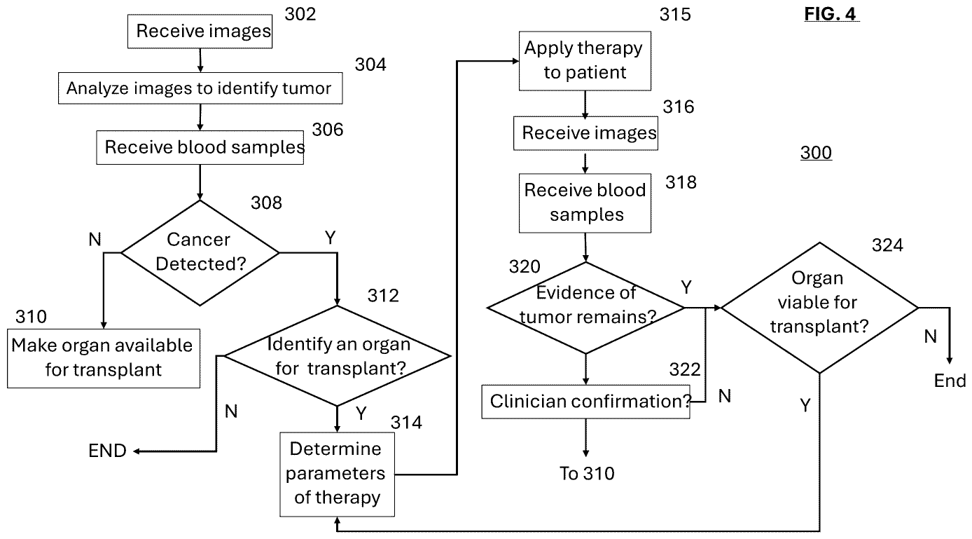

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism.” FIG. 3 illustrates an ultrasound pulse that can be used for shock scattering histotripsy. As shown the ultrasound pulse can include a leading negative half cycle, a peak positive half cycle, a peak negative half cycle, and a trailing peak positive half cycle (with the pulse traveling from right to left on the page). As shown, the trailing peak positive cycle has a lower amplitude than the peak positive cycle. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface if the amplitude of those cycles is sufficient, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When the amplitude(s) of positive half cycle(s) of each pulse are limited, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism.”

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency,” “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Organ Transplant

As described above, one aspect of the disclosure is directed to increasing the volume of organs available for transplant to patients in need. One of the major breakthroughs in modern medicine is the ability to transplant organs from one patient to another. The first successful kidney transplant occurred in 1954, and liver, heart, and pancreas transplants were first achieved by the late 1960s. Heart and lung transplants were not successful until the 1980s and the first adult-to-adult living donor transplant was not achieved until 1998. One of the original difficulties for organ transplantation was the disconnect between the location of the needy recipient and location of the donor organ. Often this meant that implantable organs were unclaimed as the potential recipient was not known to the hospital with the donor organ. Or the recipient was located too far from the donor. Modern technologies including tele-communications and internet databases have substantially eliminated the disconnect between donors and patients in need. Indeed, in 2022 a historic milestone was achieved with 1 million organ transplants having been completed in the United States, more than any other country in the world. Moreover, newly developed ex vivo perfusion techniques enable the organs to be kept viable for implantation for many more hours after removal from the donor patient than had been previously possible. These perfusion techniques have greatly increased the time available to bring the donor organ and the patient in need together.

Despite these incredible advances, or in part because of them, many patients in need are still unable to secure a donor organ before their disease state renders them unfit for transplant or in some cases the potential recipient succumbs to their disease. It is an instance where demand far outstrips supply. And the difference between demand and supply is expected to increase in the near future.

The United Network for Organ Sharing (UNOS) originated in the 1970's and sought to develop a computerized system for matching donors with transplant candidates. UNOS was awarded the first contract of what became the Organ Procurement & Transplantation Network (OPTN). OPTN links all professionals involved in the U.S. organ donation and transplantation system. Among OPTN's missions is to improve U.S. systems so that more life-saving organs are available for transplant. As part of that improvement OPTN publishes a variety of educational materials including a list of reasons that organs are refused for transplantation. While some of these reasons relate to logistics and other technical issues, a grouping of the bases for rejection are under the heading of “Disease Transmission Risk.” The transmissible diseases include everything from an infection (e.g., meningitis), to blood borne diseases (e.g., HIV), to actual or suspected malignancy with the organ.

This disclosure is directed to utilization of histotripsy system 100 to treat diseased or damaged organs and render them fit for transplantation. Histotripsy is a unique therapy, particularly among current cancer treatments of surgery, radiation, and chemotherapy. As noted above, unlike surgical approaches, histotripsy requires no incisions into the patient. Like radiation, histotripsy is preferably performed as an extracorporeal procedure, meaning that it can be performed entirely from outside the body. However, unlike radiation, the application of ultrasound energy via histotripsy does not damage any of the tissues of the patient other than at the focus of the therapy transducer 102. As a result, none of the patient's tissue from the skin to the intended target, which are not at the focus of the ultrasound energy are adversely affected by the therapy and therefore not damaged during the therapy. Moreover, as compared to systemic therapies such as chemotherapy, only the necessary tissue receives the therapy. As a result of the high level of localization of histotripsy therapy, the patient experiences no pain and only very localized tissue trauma at the intended target (e.g., tumor or lesion). Indeed, often patients comment after receiving histotripsy therapy that they are unaware that any procedure has been undertaken and need assurances from the medical staff that they have indeed received treatment. Further, unlike radiation and chemotherapy, there are no negative systemic effects from histotripsy including nausea, hair loss, swelling, burns, or becoming immuno-compromised. This means that the many patients who might be ineligible for receiving one or more of the other therapies are in fact eligible for histotripsy therapy.

The lack of systemic trauma to, or pain experienced by, the patient renders histotripsy a unique therapy for use in treating diseased organs prior to harvesting and while still within the patient or more specifically, donor patient. While patients and family members are typically unwilling to subject themselves or loved ones to additional pain and suffering or prolong the time that the patient is on life supporting systems, the lack of pain or other negative side effects from histotripsy, make it possible to still honor the patient's intentions that their organs be made available to others.

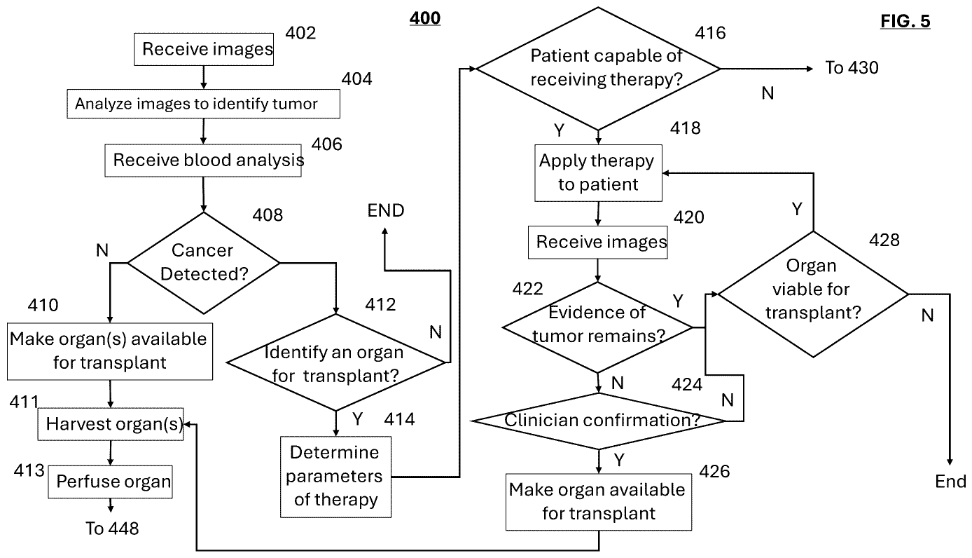

An exemplary method 300 for treating an organ in connection with this application in FIG. 4. These steps may be performed individually or as part of a software application focused on donor organ identification, therapy, recovery, and implantation. At step 302 an initial set of imaging is received (e.g., from CT, MRI, Cone beam CT, fluoroscopy, and others). The imaging may be analyzed (both using neural networks or AI and manually) to identify potential tumors or lesions in one or more of the organs of the patient at step 304. In parallel with the image analysis, blood samples are collected and analyzed and the results of the analysis is provided (e.g., to an organ donation application) at step 306. At step 308 a determination is made whether cancer(or other non-transplantable conditions or disease state) is detected in at least one organ of the patient through analysis of the images and the blood screening results, if no cancer is detected in a particular organ, it can be flagged as available for transplant at step 310, and made available to a recipient following standard protocols as set forth by UNOS.

However, if at step 308 a tumor is found, the method progresses to step 312 where utilizing the image analysis and the blood testing and biomarker analysis, the application, for example using a matrix of criteria seeks to identify at least one organ of the patient that though currently likely to be rejected from potential for implantation under the OPTN criteria, may be treated with histotripsy and rendered implantable. The matrix of criteria for the application may include size of tumor or lesion in organ, shape of the tumor or lesion, density and make-up of the tumor or lesion, stage of the cancer in that and other organs, metastases in other organs, age of the patient, other health factors of the patient, related disease states of the organ, health of the patient's immune system, and other factors. If no such organ is identified at step 312, the method ends.

Though described herein in connection with identification of tumors at step 308, the disclosure is not so limited. Step 308 may include identification of any defect which might result in the rejection of an organ for use in transplant procedures. As an example, where a patient is experiencing cirrhosis of the liver, where normal liver tissue is replaced by scar tissue and regenerative nodules, that liver would historically been prevented from being considered for potential transplant. However, in accordance with the disclosure the use of histotripsy may render the cirrhotic liver, or a suitable portion thereof, eligible for transplant. Other organ disease states may similarly receive histotripsy therapy and be rendered eligible for transplant. Accordingly, though step 308 specifically refers to determination of the presence of cancer, the disclosure is not so limited, and steps 308 and 312 may generally refer to determination of whether a defect detected in an organ or elsewhere within the patient, can be eliminated to render the organ potentially eligible for transplant.

With the goal of preserving at least the one identified organ for transplantation, histotripsy therapy is applied to the tumor or lesion in the organ. However, prior to application of therapy, at step 314 the parameters of the therapy must be determined for the particular tumor(s) or lesion(s), the organ, and the patient. A variety of factors such as number and size of the lesions/tumors may be considered, but also whether the application of therapy is to emulsify the entire tumor(s) or lesion(s) or to emulsify just a portion of the tumor(s) or lesion(s). The duration of the procedure, complicating factors such as patient anatomy and geometry (e.g., location of ribs/other hard tissues), treatment depth, position of patient for therapy delivery, and others. Some of the factors related to the planning of the histotripsy therapy are described in greater detail in commonly assigned and co-pending U.S. patent application Ser. No. 18/642,529, filed on Apr. 22, 2024 titled HISTOTRIPSY SYSTEMS AND ASSOCIATED METHODS INCLUDING USER INTERFACES AND WORKFLOWS FOR TREATMENT PLANNING AND THERAPY, the entire contents of which is incorporated herein for all aspects of the planning of therapy and the application of treatment.

Similarly, as there may be more than one tumor or lesion in the organ, a determination to treat all or a select number less than all of the tumors or lesions may be required. As is known, the use of histotripsy in a patient triggers an immune response that allows the human body to produce T-cells, and other immune responses that actively target cancer cells. It has been observed in prior analyses of histotripsy that not just the cancer cells within the organ are attacked and killed by these T-cells but also cancer cells in other organs are attacked and killed by the T-cells and eliminated from the body by the immune response following the application histotripsy to at least one tumor within the body. The number and completeness of the emulsification of the tumor(s) via histotripsy may be based on a number of factors including the age of the patient, the robustness of their immune system, other immune responses on going and observable in the blood samples, and other factors. In some cases, the application may have access to historical data sets defining outcomes for application of therapy to prior patients (e.g., big data sets analyzed by neural networks) to provide analytical and statistical data on the likely outcomes from full or partial treatment of the tumor(s). These analyses can be utilized by the clinician in determining the course of therapy application for the particular patient. At step 315 the desired therapy is applied to the tumor(s) or lesion(s) as desired by the surgeon in combination with the software application and all of the collected data (e.g., images, blood work, AI input, etc.).

With the histotripsy therapy applied to the tumor of the organ, and optionally following a period of time for blood chemistry to stabilize, follow-up imaging can be acquired at step 316 and blood samples acquired at step 318. At step 320 utilizing image analysis and blood testing a determination can be made whether the tumor(s) or lesion(s), or evidence of them remain in the organ. In no evidence of the prior tumor(s) or lesion(s) is found, the application can present such indication on one or more user-interfaces associated with the application. At step 322 the clinician can confirm the elimination of the tumor(s) or lesion(s) from the organ, and the method returns to step 310 and the organ can be made available to a recipient following standard protocols as set forth by UNOS. If the determination at steps 320 or 322 is that there remains evidence of the tumor(s) or lesion(s) the method advances to step 324 where a determination is made whether the organ(s) remains viable for eventual transplant. The determination at step 324 may be undertaken using a similar or the same criteria matrix of step 312. Additional factors may be considered including efficacy of the therapy, percentage or magnitude of change of the tumor(s) or lesion(s), levels or magnitude of immune response resulting from the therapy (e.g., as observed in the blood samples), as well as other factors associated with the health of the patient and the organ. If the determination is that the organ can no longer be made viable for transplant, then the method ends. However, if the organ remains viable for eventual transplant, the method returns to step 314 where therapy parameters of the further therapy are determined, and the method continues as described above until one of the end points is achieved.

In method 300 described above, the organ donor is likely someone with a terminal illness and has some months live. As will be appreciated, patients with cancers are currently excluded from being donors due to the risk of spreading the cancer to an organ recipient. Following the method the combination of applied histotripsy therapy and immune response to effectively eliminate the tumor(s) or lesion(s) in an organ of the patient so that the organ can satisfy the criteria for transplantation as set forth by OPTN. This can be done without inducing any of the negative side effects associated with current therapies. In this manner the pool of potential organ donors can be increased which in turn increases the supply of transplantable organs.

Method 300 is generally intended for treating diseased organs, however, the same methodology could be used to improve healthy organs. In one example, the donor receives histotripsy in either the organ intended for donation or another organ, both of which may be healthy, but for the purpose of triggering an immune response within the patient. The immune response helps ensure that any disease that might be at undetectable levels is attacked and eliminated by the T-cells generated as part of the immune response. In this manner, the health of the organ can be potentially improved as compared to prior to inducement of the immune response.

While method 300 indeed yields additional organs for transplant from donors having weeks or even months of life expectancy (e.g., those on hospice care) and provides those organs to patients in need, the disclosure is not so limited. Another aspect of the disclosure is related to employing histotripsy in an emergency setting or another setting where the patient is either about to expire or has only recently expired. Often in the emergency setting there is little to no time to allow for the triggering of an immune response to assist in treating the donor and any imperfections in the organ(s). In a further method 400 of the disclosure, a donor presents themselves, for example, at an emergency room having suffered from some medical condition for which though treatment is attempted nonetheless proves fatal to the patient. At step 402 images (e.g., CT, MRI, etc.) images are captured of the patient. The imaging may be analyzed (both using neural networks or AI and manually) to identify potential tumors or lesions in one or more of the organs of the patient at step 404. In parallel with the image analysis, blood samples are collected and analyzed and the results of the analysis is provided (e.g., to an organ donation application) at step 406. At step 408 a determination is made whether cancer is detected in at least one organ of the patient through analysis of the images and the blood screening results, if no cancer is detected in a particular organ, it can be flagged as available for transplant at step 410, and made available to a recipient following standard protocols as set forth by UNOS. At step 411, the organ(s) can be harvested and at step 413 the organ can be perfused to preserve tissue health and the viability of the organ for transplant into an identified recipient.

However, if at step 408 a tumor is found, the method progresses to step 412 where utilizing the image analysis and the blood testing analysis, the application, for example using a matrix of criteria seeks to identify at least one organ of the patient that though currently likely to be rejected from potential for implantation under the OPTN criteria, may be treated with histotripsy and rendered implantable. The matrix of criteria for the application may include size of tumor or lesion in organ, shape of the tumor or lesion, density and make-up of the tumor or lesion, stage of the cancer in that and other organs, metastases in other organs, age of the patient, other health factors of the patient, related disease states of the organ, health of the patient's immune system, and other factors. If no such organ is identified at step 412, the method ends.

As described above, though cancer detection is described in step 408, any defect of the organ that can be treated with histotripsy can be detected and analyzed at steps 408 and 412 without departing from the scope of the disclosure.

With the goal of preserving at least the one identified organ for transplantation, histotripsy therapy is applied to the tumor or lesion in the organ. However, prior to application of therapy, at step 414 the parameters of the therapy must be determined for the particular tumor(s) or lesion(s), the organ, and the patient. In accordance with method 400, the parameters for treating an organ may be simplified to identification of the size (plus a specified margin) to achieve complete treatment and emulsification of the tumor(s) or lesion(s). Other parameters that might be considered in connection with the therapy application are the positioning of the patient needed achieve the therapy, intervening hard tissues such as ribs, and other physical limitations of the patient that might impede therapy.

At step 416 a determination is made whether the patient is capable of receiving histotripsy therapy in their current condition. As will be appreciated, other medical limitations, beyond the existence of tumors or lesions, may prevent the organ(s) identified at step 408 from receiving histotripsy. When a patient expires, the organs of the patient have a relatively short time frame before they are no longer viable for implantation. Accordingly, the time between passage of the patient to perfusion of the organ to preserve it for transplantation must be shortened as much as possible. Further, as will be appreciated, particularly in emergency situations, despite the lack of any pain or other negative side effects from histotripsy, the patient may not be in any condition to have a coupling assembly 212, and filed with a coupling medium, placed on their body and to undergo the application of histotripsy. Accordingly, and without departing from the scope of the disclosure, method 400 may re-order or skip one or more steps without departing from the scope of the disclosure.

If at step 416 it is determined that histotripsy therapy can be applied to the patient, at step 418 histotripsy therapy is applied to the organ of the patient in accordance with the parameters established at step 414. At step 420 follow-up imaging can be acquired. At step 422 utilizing image analysis a determination can, be made whether the tumor(s) or lesion(s), or evidence of them remain in the organ. In no evidence of the prior tumor(s) or lesion(s) is found, the application can present such indication on one or more user-interfaces associated with the application. At step 424 a clinician can confirm the elimination of the tumor(s) or lesion(s) from the organ, and the method advances to step 426, where the organ can be identified as available for donation and ultimately transplant following standard protocols as set forth by UNOS. With this identification on the demise of the patient, the now treated organ can be harvested at step 411 and optionally perfused at step 413 to preserve tissue health and the viability of the organ for transplant into an identified recipient and the method continues at step 448 (described below). If, however, the clinician does not confirm the elimination of the tumor(s) or lesion(s) a determination is made at step 428 to determine whether to apply further therapy or reject the organ. If the organ is rejected to method ends but if further treatment is possible the method returns to step 418 for further application of histotripsy therapy.

If, however, at step 416 it is determined that histotripsy cannot currently be applied to the patient, as may be the case due to injury or disease state, then harvest of the organ must await the passing of patient. At step 430, following the unfortunate demise of the patient, the identified organ is harvested in accordance with all appropriate parameters to enable transplant. At step 432, the organ may be perfused to maintain its viability for transplant for as long as possible. At step 434 a determination is made as to whether to treat the harvested and perfused organ immediately or after implantation. If the determination is to treat immediately, the harvested and perfused organ is treated at step 436 to emulsify the tumor(s) or lesion(s) within the organ in accordance with the parameters set forth in step 414. At step 438 imaging is acquired and analyzed to determine the efficacy of the treatment (e.g., via neural networks, AI, or manually). At step 440 an indicator of the efficacy of the therapy can be presented on a user interface and the images of the treated organ along with other information presented to a clinician for review and confirmation at step 442. If confirmed at step 442 the method advances to step 444, where the organ can be identified as available for donation and ultimately transplant following standard protocols as set forth by UNOS. If, however, the clinician does not confirm the elimination of the tumor(s) or lesion(s) a determination is made at step 446 to determine whether to apply further therapy or reject the organ. If the organ is rejected to method ends but if further treatment is possible the method returns to step 436 for further application of histotripsy therapy.

Referring back to step 434, if the determination is made to treat after implantation, the harvested and perfused organ, following standard protocols is prepped for transplant, transported to the patient in need, and transplanted at step 448. Following the transplant an inquiry is made at step 449 whether the organ(s) require therapy. If therapy has already been applied (e.g., step 418) or therapy is required (e.g. steps 408 and 410) then the method end, however, if the now implanted organ(s) require therapy, images of the recipient and the donor organ are acquired at 450 and at 452 parameters for the application of histotripsy therapy are be determined for the tumor(s) or lesion(s), the organ, and the patient utilizing the same or similar analysis as the determination of the therapy parameters at step 414. With the parameters for therapy determined, histotripsy therapy is applied at step 454 to the patient. Though described herein as occurring sequentially and at approximately the same time as the implantation of the harvested organ, those of ordinary skill in the art will recognize that the application of histotripsy therapy to the now implanted organ (e.g., steps 450-454) may occur weeks and even months after the implantation of the harvested organ to allow the recipient patient time to heal from the surgery. Such time may also allow for the weaning from and the elimination from the patient of immunosuppressant drugs employed during transplant.

Following therapy application, for example two weeks after application of the histotripsy therapy, images of the implanted organ are acquired at step 456. The imaging may be analyzed (both using neural networks or AI and manually) to assess the efficacy of the therapy at step 458. In parallel with the image analysis, blood samples are collected and analyzed at step 460. At step 462 an indicator of the efficacy of the histotripsy therapy can be presented on a user interface along with the images of the treated organ and with other information (e.g., the immune response from the blood samples). This clinician can review the information and confirm the findings at step 464. If confirmed at step 464 the method end and the patient enters a watchful waiting period similar to current transplant patients, where periodically imaging and blood analyses are performed, along with others to monitor the status of the patient and the transplanted organ. In certain circumstances, based on the findings during the watchful waiting period, the patient may receive a subsequent histotripsy treatment. This subsequent histotripsy treatment may be weeks and even months later. The additional histotripsy treatment may ensure that the newly transplanted organ(s) is healthy and remains free of the disease necessitating the treatment, and to remove or treat any unhealthy tissue. In some instances, the subsequent histotripsy treatment may be performed on the same organ, however, the subsequent histotripsy treatment may be performed on a different organ including in healthy tissue. Histotripsy treatment is associated with a systemic immune response and has been shown to trigger a t-cell response from the body to eliminate diseased tissue in untreated and even unrelated (e.g., different organs) of the body.

If, however, at step 464 the clinician is not satisfied with the histotripsy efficacy determination, at step 466 a determination is made whether more therapy can be applied to the organ and patient. If no further histotripsy can currently be performed the method ends, however, if further treatment can be applied to the patient and the transplanted organ, the method returns to step 454 for application of further therapy.

In accordance with aspects of the disclosure, because the immune response to histotripsy is focused on cancers found in the donor organ, the application of histotripsy may result in a focused immune response targeting just the cancer cells of the type found in the donor organ, thus histotripsy therapy may be applied at any time after transplant to ensure that any cells beyond the treated tumor(s) or lesion(s) are killed by the T-cells of the patient receiving the donor organ.

As will be appreciated, by applying histotripsy to the organ after transplantation and triggering an immune response may be repressed when following transplant most recipient patients receive immunosuppressant drugs to prevent the rejection of the transplanted organ. Accordingly, without departing from the scope of the disclosure, the application of histotripsy therapy to the organ in the recipient patient may be delayed until after the cessation of use of immunosuppressant drugs by the patient receiving the donor organ.

As is known, certain organs are regenerative and when afforded an opportunity to heal can regrow to their original size and function. One such organ is the liver, another in certain circumstances is the pancreas. Similarly, other organs can maintain most, if not all, of their functionality even when a significant portion of the organ is resected. As an example, the pancreas retains typically just the head following a Whipple procedure, yet much of the functionality of the pancreas remains despite the removal of large portions of the organ.

By managing the focal field of the therapy transducer 102, a tissue selective field is generated. This tissue selectivity ensures that the treatment of tumors or lesions having a larger blood vessel or other duct (e.g., gall duct, bile duct, ureter, etc.) histotripsy is capable of eliminating the tumor, without damaging the blood vessel or duct. Though tumors are typically treated by creating small, generally ovoid shaped, focal fields of ultrasound energy that emulsify the tissue in that focal field. For a given tumor or lesion tens or even hundreds of these small ovoid focal fields are generated to emulsify the tissue of the tumor or lesion and some margin. However, the therapy transducer 102 is not so limited and its drive signal and focal field can be adjusted to generate an ultrasonic scalpel field. The therapy transducer 102, generating the ultrasonic scalpel field is capable of ex vivo cutting tissue within the patient (e.g., the organ donor). This ultrasonic scalpel field remains very tissue specific.

In a further aspect of the disclosure, rather than treating cancerous tumors or lesions within an organ, histotripsy is employed to perform highly selective resection of organs such as the liver or the pancreas within the body of the donor (e.g., step 418 of method 400). Thus, for example, when an organ from a potential donor is found to have one or more tumors or lesions in one portion of the organ, but a sufficiently sized portion of the organ is free of tumors or lesions histotripsy can be employed to resect the healthy portion of the organ from the diseased portion. Due to the high tissue selectivity blood vessels and ducts along the resection line are largely unaffected by the resection of the organ. In this manner the organ functions can continue when the organ being treated is still in the donor patient (e.g., method 300). Regardless of when histotripsy is applied (e.g., before or after harvesting and perfusion), resection of the healthy portion of the organ from the diseased portion with the ultrasonic scalpel field creates a line of dead and emulsified tissue along the resection line. This emulsified tissue can be washed away after resection, e.g., using saline solution or another sterilized surgical solution to expose the blood vessels and ducts within the organ along the resection line.

Once revealed, all of the blood vessels and ducts along the resection line may be ligated either via manual surgical procedures (e.g., via sutures and clamps) or via electrosurgical or mechanical ultrasonic vessel ligation tool to seal and sever the blood vessels of the healthy portion of the organ. The healthy portion of the donor organ can then be made available for donation and ultimately transplant following standard protocols as set forth by UNOS.

The use of histotripsy and an ultrasonic scalpel for resection of a healthy portion of an organ to a diseased portion of an organ can be undertaken in connection with either method 300 or method 400. Further, the resection may be made in conjunction with the treatment of one or more tumors or lesions in an effort to trigger the immune response and the generation of T-cell to seek out and kill any unidentified cancerous cells, tumors, or lesions.

Still further, though described in connection with separating healthy tissue from diseased tissue, the disclosure is not so limited. For example, in the context of lungs, while removing damaged lung segments from healthy might be one use of the ultrasonic scalpel, the disclosure is not so limited. Lungs come in pairs, but not every transplant requires both lungs. A further aspect of the disclosure is separating the right and left lungs so that one healthy pair of lungs could be utilized to benefit two patients in need without departing from the scope of the disclosure. As will be appreciated, other organs, particularly those that are found in pairs, including kidneys and eyes, can benefit multiple patients in need. Further, in some instances it may be possible for a single liver to benefit multiple patients in need.

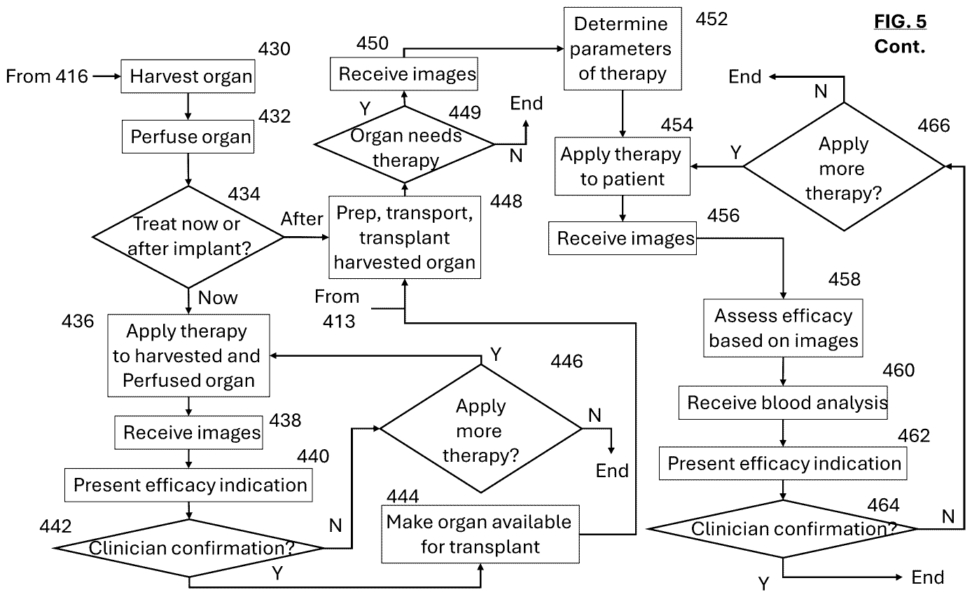

As described herein, and as depicted in FIGS. 4 and 5, one disease state that is identified and treated is the presence of cancerous tumors or lesions within the organ identified for therapy and ultimately transplant. However, the disclosure is not so limited and as suggested above, the therapy may be employed for a variety of disease states including the presence of benign tumors, cirrhosis, cysts, polyps, trauma, and others without departing from the scope of the disclosure.

As described elsewhere herein, this disclosure contemplates at least four scenarios for the acquisition of and the treatment of a disease state in a potential organ for transplant that would otherwise be rejected. In one scenario, a potential donor who is nearing their end of life (e.g., on hospice care) receives histotripsy therapy to one or more organs while they remain in the patient. A second scenario is an emergency donor patient (e.g., from a car crash or other accident) whose condition while fatal could be sustained for some duration to allow for application of in vivo histotripsy therapy of one or more organs. Such an action may be effective, for example, where the patient is ruled to be brain dead from their accident but is otherwise whole such that histotripsy could be administered to the patient. A third scenario contemplated by the methods described herein is the application of histotripsy to harvested and perfused organs. Often, particularly in an emergency situation the patient expires or their injuries are such that the patient could not undergo histotripsy or the benefits of in vivo histotripsy (e.g., immune response) are unlike to be triggered. As a result, with the expiration of the patient, the organs are immediately harvested and to preserve them for implantation the organs are perfused (e.g., blood flow and other fluids induced into the organ post-harvest). As a result, the histotripsy therapy is applied to the harvested organ and not to the donor patient. Still a fourth scenario contemplated by the methods described herein is the harvesting and implantation of a diseased organ into a recipient patient in need. After implantation of the diseased organ, the organ now within the recipient patient is treated with histotripsy therapy to reduce and eliminate the disease state of the implanted organ.

In accordance with this disclosure, organs that would otherwise be unable to be identified, cleansed of their disease, and then implanted into a needly recipient. Thus, using histotripsy the quantity of organs available for transplant can be greatly increased to meet the demand that as noted above is expected to increase in the near future.

EXAMPLES—the methods described in this disclosure are beneficially considered in connection with the following prophetic examples:

Example 1—a diseased organ is harvested from a donor. The diseased organ is transplanted into a recipient patient. Following transplantation of the diseased organ the diseased organ is treated in vivo in the recipient patient with histotripsy therapy. The treatment of the diseased organ triggers an immune response within the recipient patient. The immune response assists in treating the disease state of the diseased organ as well as healing wounds resulting from the transplantation of the diseased organ in the recipient patient.

Example 2—a potential donor having one or more diseased organs is identified. For example, the potential donor may be in hospice, in a medically induced coma, or another responsive or unresponsive state. The one or more diseased organs are treated in vivo in the potential donor with histotripsy therapy. Blood biomarkers of the patient are monitored and imaging is acquired of the diseased organ. Based in part on a change in blood biomarkers and observed disease state in the images the one or more organs are deemed eligible for transplant following a histotripsy treatment. After the passing of the patient, the newly eligible organ is harvested from the donor patient made available for transplant in a recipient patient.

Example 3—a diseased organ is harvested from a donor. The harvested organ is perfused to maintain its viability for transplant. The harvested and perfused diseased organ is treated with histotripsy therapy. After treatment, the harvested organ is imaged to confirm efficacy of the histotripsy therapy, and if efficacy is confirmed the harvested organ is made eligible for transplant. The donor organ is transplanted into the recipient patient. Optionally, after transplantation of the donor organ, the recipient patient is treated with histotripsy therapy, the treatment of the recipient patient may be applied to the transplanted organ or may be another organ of the recipient patient. The histotripsy therapy applied to the recipient patient can be applied to the transplanted organ or to another organ (e.g., a healthy organ). The histotripsy therapy triggers an immune response within the recipient patient to avoid rejection of the transplanted organ and promote healing from the transplant surgery.

Example 4—a diseased organ is harvested from a donor. The harvested organ is perfused to maintain its viability for transplant. The diseased and perfused organ is analyzed (e.g., via imaging analysis) to confirm that certain criteria are satisfied (e.g., no more than 3 tumors of less than 3.5 cm in diameter). With the criteria satisfied and the harvested donor organ perfused, histotripsy therapy is applied to the donor organ to eliminate or reduce the disease state of the organ. After application of therapy, a determination is made whether a second criteria is satisfied (e.g., blood biomarkers of the disease fall below a threshold, treatment of all tumors confirmed by imaging) the harvested donor organ is made available for transplant into a recipient patient.

Example 5—a diseased organ is harvested from a donor. The harvested organ is perfused to maintain its viability for transplant. A portion of the harvested organ is skeletonized to isolate healthy tissue from diseased or damaged tissue of the harvested organ but retain the patency of any blood vessels or ducts extending through the skeletonized portion of the harvested organ. The diseased portion of the harvested organ is resected from the healthy portion of the harvested organ to seal the blood vessels and other ducts or fluid passageways. The healthy portion of the diseased organ is then transplanted into the recipient patient.

Example 6—a deceased potential organ donor (e.g., a cadaver) is identified. One or more potential organs for donation are identified within the decedent. Each of the one or more organs may optionally have to satisfy some criteria (e.g., no more than 3 tumors of less than 3.5 cm in diameter). Determining that the one or more organs satisfy the criteria is undertaken with analysis of images of the one or more organs. The organs that satisfy the criteria are perfused to maintain their viability for transplant. The perfusion of the one or more organs may be undertaken within the body of the deceased or external to the body of the deceased but functionally connected to the other organs, blood vessels, and fluid ducts of the decedent. The one or more now perfused organs are treated with histotripsy therapy. After application of therapy, a determination is made whether a second criteria is satisfied (e.g., blood biomarkers of the disease fall below a threshold, treatment of all tumors confirmed by imaging) the one or more organs are harvested and made available for transplant into a recipient patient.

The embodiments described above are cited by way of example, and that the present invention is not limited to what has been particularly shown and described hereinabove. Rather, the scope of this disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are considered an integral part of the application.

We claim:

1. A method of rendering a diseased organ implant eligible, comprising:
acquiring an image data set of a potential donor organ;
identifying one or more disease states in the potential donor organ, wherein the one or more disease states render the organ ineligible for transplant;
harvesting the potential donor organ;
applying histotripsy therapy; and
confirming at least one of elimination or/reduction of the disease state in the potential donor organ.

2. The method of claim 1, wherein the histotripsy therapy is applied prior to harvesting of the potential donor organ.

3. The method of claim 1, wherein histotripsy therapy is applied after harvesting of the potential donor organ.

4. The method of claim 3, further comprising performing perfusion on the potential donor organ prior to or during the application of the histotripsy therapy.

5. The method of claim 4, further comprising assessing one or more blood biomarkers to assess an efficacy of the histotripsy therapy to confirm the at least one of elimination or reduction of the disease state.

6. The method of claim 1, wherein applying histotripsy therapy skeletonizes a portion of the potential donor organ.

7. The method of claim 6, wherein the skeletonization reveals one or more blood vessels or ducts within the potential organ.

8. The method of claim 7, further comprising ligating the one or more blood vessels or ducts within the potential organ.

9. The method of claim 1, wherein the histotripsy therapy is applied to a tumor or cyst.

10. The method of claim 9, wherein at least one of a mechanical properties or integrity of the tumor or cyst are reduced following application of histotripsy therapy.

11. The method of claim 8, further comprising determining that a sub-portion of the potential donor organ, skeletonized, and ligated from the potential donor organ is suitable for transplant.

12. The method of claim 1, wherein application of histotripsy therapy triggers an immuno-response.

13. The method of claim 12, further comprising monitoring blood biomarkers to assess a magnitude of the immuno-response.

14. The method of claim 13, wherein confirming the at least one of elimination or reduction of the disease state in the potential donor organ is based on a change in the blood biomarkers.

15. The method of claim 14, further comprising acquiring the blood biomarkers before application of histotripsy therapy and after application of at least one histotripsy therapy.

16. The method of claim 1, further comprising acquiring a second image data set, wherein confirming at least one of elimination or reduction of the disease state in the potential donor organ is based on a comparison of the image data set and a second image data set.

17. The method of claim 16, wherein an artificial intelligence or a trained neural network performs the comparison of the image data set and the second image data set.

18. The method of claim 1, wherein the potential donor organ is selected from the group consisting of a liver, a kidney, bone marrow, a lung, a pancreas, an esophagus, an eye, a pituitary gland, a trachea, skin, and blood.

19. The method of claim 1, wherein the disease state includes one or more of cancerous tumors, cirrhosis, cysts, fatty deposits, or blood cancers.

20. The method of claim 1, wherein the histotripsy therapy is applied to a diseased portion of the potential donor organ.

21. The method of claim 1, wherein the histotripsy therapy is applied to a non-diseased portion of the potential donor organ.

22. The method of claim 1, wherein the histotripsy therapy is applied to a diseased portion of an organ other than the potential donor organ.

23. The method of claim 1, further comprising implanting the potential donor organ in a patient in need thereof.
